(19) United States
(12) Patent Application Publication
(10) Pub. No.: US 2023/0166002 A1
(43) Pub. Date: Jun. 1, 2023

(54) METHODS AND COMPOSITIONS FOR PERIODONTAL AND SOFT TISSUE REGENERATION IN DIABETIC SUBJECTS

(71) Applicant: Khalid Alhezaimi, Newport Beach, CA (US)

(72) Inventor: Khalid Alhezaimi, Newport Beach, CA (US)

(21) Appl. No.: 18/072,545

(22) Filed: Nov. 30, 2022

Related U.S. Application Data
(60) Provisional application No. 63/284,768, filed on Dec. 1, 2021.

(51) Int. Cl. A61L 27/36; A61L 27/24; A61L 27/54

(52) U.S. Cl. CPC A61L 27/3608; A61L 27/24; A61L 27/54

(57) ABSTRACT

Provided are methods and compositions for maxillofacial and/or periodontal bone repair in hyperglycemic and/or diabetic subjects.

### CROSS REFERENCE TO RELATED APPLICATIONS

This application claims priority under 35 U.S.C. § 119 to U.S. Provisional Application Ser. No. 63/284,768, filed Dec. 1, 2021, the disclosures of which are incorporated herein by reference.

### FIELD OF THE INVENTION

The disclosure generally concerns methods of tissue engineering, regeneration and repair, and more particularly relates to methods and compositions for treating diabetic subjects having periodontal, maxillofacial and tissue damage.

### BACKGROUND

The use of growth factors (GFs) as adjuncts to conventional guided bone regeneration (GBR) and guided tissue regeneration (GTR) techniques have trended as a material of choice for periodontal regeneration. The binding between GFs and cell membrane receptors of the target cells induces intracellular signaling pathways that may alter cellular activity and phenotype by gene activation.

Several studies have reported that the severity of periodontal disease is enhanced in patients with chronic hyperglycemia as compared to those with well-controlled diabetes and medically healthy controls. Persistent hyperglycemia is associated with increased formation and accumulation of advanced glycation products in the periodontal tissues that worsen inflammation and retards healing. Experimental studies have also reported that chronic hyperglycemia significantly delays osseous defect healing by augmenting inflammation and impairing proliferation of osteoblasts and periodontal ligament fibroblasts.

### SUMMARY

The disclosure provides a composition comprising allogeneic and/or xenogeneic bone graft material comprising recombinant platelet derived growth factor (rPDGF). In one embodiment, the rPDGF is recombinant human PDGF (rhPDGF). In another or further embodiment, the bone graft material is xenogeneic to a subject. In still another embodiment, the bone graft material is allogeneic to a subject. In yet another embodiment, the allogeneic and/or xenogeneic bone graft material comprises allogeneic and xenogeneic material. In another embodiment, the allogeneic and/or xensogeneic bone graft material comprising rPDGF further comprises one or more additional factors. In a further embodiment, the one or more additional factors comprise extracellular matrix materials. In still a further embodiment, the extracellular matrix materials are selected from Type I collagen, Type II collagen, Type III collagen, bovine collagen, human collagen, porcine collagen, equine collagen, avian collagen, fibronectin, alginate and any combination thereof. In another embodiment, the one or more additional factors are growth factors. In another embodiment, the one or more additional factors are antibiotics, antifungals or a combination thereof.

The disclosure also provides a method of treating a bone injury in a subject with hyperglycemia, comprising administering a composition comprising allogeneic and/or xenogeneic bone graft material comprising recombinant platelet derived growth factor (rPDGF) of the disclosure. In one embodiment, the subject has hyperglycemia. In a further embodiment, the subject has chronic hyperglycemia. In another or further embodiment, the subject has diabetes. In still a further embodiment, the diabetes is type 1 and/or 2 diabetes. In another embodiment, the bone injury is the result of a periodontal infection. In another embodiment, the bone injury is a maxillofacial bone injury. In still a further embodiment, the maxillofacial bone injury is in bone surrounding the teeth (e.g., the teeth are not missing; the tooth/teeth comprise the surrounding area including the periodontal and osseous tissue) and the bone and tissue are regenerated around the teeth. In yet another or further embodiment, the bone injury is a periodontal bone injury.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-D shows a series of reconstructed micro-computed tomography (μCT) images for group treated with rhPDGF and xenograft: (A) a sagittal view showing the thickness and the vertical bone gain from the base of the notch and to the most coronal part (arrow) regenerating the dehiscence defect; (B) a mesio-distal sagittal section demonstrating the three-wall defect bone fill (arrow); (C) an axial section 3 mm from the CEJ illustrating the thickness and bone volume (arrow); (D) series of axial section (1 mm intervals) showing the buccal bone thickness and interproximal bone fill from the 1 mm below CEJ to the 1 mm coronal to root apex.

FIG. 2A-D shows histologic results of induced chronic dehiscence and three walls periodontal defect treated with rhPDGF and xenograft: (A) shows the BBT and VBH (arrow, X4); (B) no clear transition between regenerated and adjacent bone (X8); (C-D) florescent light illustrating the vertical insertion of connective tissue fibres into the cementum and abundant amount of connective tissue fibres connecting to the tooth cementum (toluidine blue/pyronin G; original magnification X32). RD: root dentin.

FIG. 3A-D provides a series of reconstructed μCT images for group treated with EMD and xenograft: (A) a sagittal view showing absence of regenerated bone from the base of the notch and to the most coronal part (arrow) of the dehiscence defect; (B) just one case showed a 1 mm bone thickness at the base of the notch and complete absence of VBH (arrow); (C) a mesio-distal sagittal section demonstrating the three-wall defect complete lack bone fill (arrow); (D) series of axial section (1 mm intervals) showing the buccal bone thickness only in the last apical 2 mm.

FIG. 4A-F shows histologic results of induced chronic dehiscence and three walls periodontal defect treated with EMD and xenograft: (A-C) absence of VBH beyond the basal notch (arrow, X4-8) and long junctional epithelium type of attachment; (D-E) fluorescent light at higher magnification (X32) demonstrating majority of cases had vertical connective tissue fibres insertion except in once case (D) where fibres were parallel; (E-F) the connective tissue fibres attached to the cementum were very few (arrows).

### DETAILED DESCRIPTION

As used herein and in the appended claims, the singular forms “a,” “an,” and “the” include plural referents unless the context clearly dictates otherwise. Thus, for example, reference to “a protein” includes a plurality of such proteins and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Guided bone regeneration (GBR) and guided tissue regeneration (GTR) are dental surgical procedures that use barrier membranes to exclude epithelium and direct the growth of new bone and periodontaltissue at sites with insufficient volumes or dimensions of bone or periodontal tissues for proper function, esthetics or prosthetic restoration. Guided bone regeneration typically refers to ridge augmentation or bone regenerative procedures; guided tissue regeneration typically refers to regeneration of periodontal attachment.

Guided tissue regeneration is focused on development of hard and soft tissues of the periodontal attachment. At present, guided bone regeneration is predominantly applied in the oral cavity to support reconstitution of a new hard, osseous mineralized tissue growth on an alveolar ridge to allow stable placement of dental implants.

The disclosure provides compositions and methods for treating bone and tissue injury in a subject having hyperglycemia, the method comprising applying to a site of bone and/or tissue injury a composition comprising a biocompatible material and an effective amount of platelet-derived growth factor (PDGF). In one embodiment, the bone and/or tissue injury is surrounding a tooth or teeth in the subject. In one embodiment, the PDGF is PDGF-BB. In one embodiment, the biocompatible material is natural bone material. In another embodiment, the biocompatible material comprises PDGF. In still another embodiment, the biocompatible material is soaked in PDGF under conditions wherein the PDGF is adsorbed or absorbed by the biocompatible material. In another embodiment, the PDGF is a recombinant PDGF. In still a further embodiment, the recombinant PDGF is recombinant human PDGF (rhPDGF). In another embodiment, the biocompatible material is xenogeneic to the subject to be treated. In another embodiment, the biocompatible material is allogeneic to the subject to be treated. In another embodiment, the biomaterial is alloplastic (e.g., an alloplastic bone graft). In still another embodiment, the subject has hyperglycemia. In still another embodiment, the subject has chronic hyperglycemia. In still another or further embodiment, the subject has diabetes. In still a further embodiment, the subject has type 1 and/or 2 diabetes. In still another or further embodiment, the bone injury is a maxillofacial bone injury and/or injury around teeth, head or neck.

“Biocompatible” refers to compounds or compositions and their corresponding degradation products that are relatively non-toxic and are not clinically contraindicated for administration into a tissue or organ.

The biocompatible material can take the form of a gel, matrix, film, or scaffold (e.g., a porous or non-porous material that can adsorb or absorb a growth factor or other active agents.

The biocompatible material may be of any material and/or shape that: (a) allows growth factors and optionally one or more additional active agents to adsorb or absorb thereto; and (b) allows cells to grow on or in the material. A number of different materials may be used to form the material, including but not limited to: polyglycolic acid (PGA), collagen (in the form of sponges, braids, or woven threads, etc.), cat gut sutures, cellulose, gelatin, or other naturally occurring biodegradable materials or synthetic materials, including, for example, a variety of polyhydroxyalkanoates. Any of these materials may be woven into a mesh, for example, to form a framework or scaffold.

The biocompatible material can be a naturally occurring alloplastic, xenogeneic or allogeneic bone preparation. For example, suitable allogeneic bone graft material or scaffolds are available under the trade names: Coreograft™ (beta-tricalcium phosphate), Corlok™, Duet™, Profuse™, Solo™, VG1™ ALIF, VG2™ PLIF, VG2™ Ramp, Vertigraft VG2™ TLIF, Graftech™ products, Grafton™ products, Cornerstone-SR™, Cornerstone™ Select, MD™ Series, Precision™, Tangent™, Puros™, Vitoss™, Cortoss™, or Healos™. In certain embodiments, the biocompatible xenogeneic or allogeneic bone graft material can be in the form of a mesh, a gauze, a sponge, a monophasic plug, a biphasic plug, a paste, or a putty. In some embodiments, the biocompatible material can further comprise extracellular material materials such as Type I collagen, a Type II collagen, a Type III collagen, bovine collagen, human collagen, porcine collagen, equine collagen, avian collagen, or combinations thereof.

In some instances the biocompatible material is formed into a shape suitable for implantation into a bone injury site. For example, the biocompatible material can be formed into a plug to be inserted into a tooth socket. The plug or other design can then be adsorbed with or absorbed with a PDGF protein (e.g., a PDGF-BB protein).

Platelet-derived growth factor (PDGF) presents as dimers of A, B and C polypeptide chains. Five isomeric forms of PDGF have been identified: PDGF-AA, PDGF-AB, PDGF-BB, PDGF-CC and PDGF-DD. PDGF receptor signalling plays a significant role in regulation of the proliferation and cells migration including osteoblasts and fibroblasts. For example, PDGF alpha-receptors (or A-type receptors) bind only to three isoforms with high affinity while beta-receptors (or B-type receptors) bind to PDGF-BB with high affinity. This may explain the variations in impact and effect of different PDGF isoforms and their function. Platelet-derived growth factor (PDGF) regulates cell growth and division. PDGF plays a role in blood vessel formation, the growth of blood vessels from already-existing blood vessel tissue, mitogenesis, i.e. proliferation, of mesenchymal cells such as fibroblasts, osteoblasts, tenocytes, vascular smooth muscle cells and mesenchymal stem cells as well as chemotaxis of mesenchymal cells. The nucleic acid sequences of PDGF-A, PDGF-B, and PDGF-C are known and the proteins have been cloned and expressed in various systems (see, e.g., HumanKine®, Proteintech; see also UniProt accession number P01127-1 PDGFB HUMAN, which is incorporated herein by reference for all purposes). In certain embodiments of the disclosure, PDGF-BB is used in the methods and compositions.

In some embodiments, the PDGF is present in the composition from about 0.01 mg/ml to about 10 mg/ml. The PDGF may be present in at any concentration within the range above. In other embodiments, PDGF is present at or between any one of the following concentrations: about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.0 mg/ml; about 2.0 mg/ml, about 3.0 mg/ml; about 4.0 mg/ml, about 5.0 mg/ml; about 6.0 mg/ml; about 7.0 mg/ml; about 8.0 mg/ml; about 9.0 mg/ml; or about 10 mg/ml. Amounts of PDGF that can be used range from about 1 ug to about 50 mg, about 10 ug to about 25 mg, about 100 ug to about 10 mg, and about 250 ug to about 5 mg.

The PDGF (e.g., PDGF-BB) may be natural or recombinantly produced. As mentioned above PDGF-B can dimerize to form PDGF-BB. When produced by recombinant methods, a polynucleotide sequence encoding a monomer (e.g., PDGF B), can be engineered into a suitable vector and inserted into a prokaryotic or eukaryotic host cells for expression to subsequently produce the homodimer (e.g. PDGF-BB). As mentioned above commercially available GMP recombinant PDGF-BB can be obtained commercially from, e.g., Chiron Corporation (Emeryville, Calif.). Other suitable sources including R&D Systems, Inc. (Minneapolis, Minn.), BD Biosciences (San Jose, Calif.), and Chemicon, International (Temecula, Calif.).

In certain embodiments, the bone injury is a periodontaltissues injury. In another embodiment, the bone injury is a maxillofacial bone injury. In some or further embodiments, the injury is the result of an infection, physical injury such as an accident or violence, or surgery. Typically the bone injury is associated with diabetes or a complication of diabetes or other hyperglycemic disease or disorder.

In yet another embodiment, the disclosure provides a method of promoting growth and/or regeneration of bone tissue at a site of bone injury in a hyperglycemic or diabetic subject in need thereof, wherein the method comprises implanting a biocompatible material comprising PDGF (e.g., PDGF-BB) to the site or injury or site to be repaired and/or regenerated. In certain embodiments, the biocompatible material comprising PDGF may further comprise additional factors to promote tissue growth and/or regeneration and/or to control infection. In certain embodiments, the site to be treated is first prepared to provide a suitable tissue bed for implantation of the biocompatible material. In other embodiments, the site to be treated is a maxillofacial bone site or periodontal tissues site. In another embodiment, the PDGF is a recombinant human PDGF. In still a further embodiment, the PDGF is PDGF-BB. In still another or further embodiment, the biocompatible material is molded to fit into or model the bone tissue to be repaired or regenerated.

In some embodiments, to promote bone regeneration, the site to be treated undergoes intramarrow bone penetration at the site to promote cells and factors from the bone marrow to migrate onto or into the biocompatible material comprising the PDGF. For example, cells selected from periosteogeneic cells, angiogenic cells, stromal cells, mesenchymal cells, osteoprogenitor cells, osteoblasts, osteoclasts, platelets and combinations thereof can migrate into or onto the biocompatible material.

As mentioned above, the biocompatible material comprising the PDGF can be alloplastic, allogeneic and/or xenogeneic cell-free bone material. In addition, the biocompatible material can be in the form of a paste, plug, sponge etc. and can be formed to fit the site of repair.

As used herein reference to “a site” or “the site”, or “sites” refer to maxillofacial periodontal site in a subject that is to be treated and/or that has bone injury or damage.

A bone injury that can be treated by the methods and compositions of the disclosure includes injuries resulting from physical trauma that can cause bone fracturing, bone crushing, bone pulverizing, pathology, tumors and the like. Such physical trauma can result from invasive medical procedures due to reconstructive surgery, periodontal surgery and the like. Such surgeries may be due to infections from fungal or bacterial, microbial or viral agents or the mouth and teeth.

As described in more detail below, the data demonstrate that the compositions of the disclosure (e.g., alloplastic, allogeneic and/or xenogeneic bone material comprising PDGF) promote the migration and/or infiltration of at least one endogenous component in hyperglycemic or diabetic subjects. Such components include periosteogeneic cells, angiogenic cells, stromal cells, mesenchymal cells, osteoprogenitor cells, osteoblasts, osteoclasts, platelets, and the like.

The effect of the methods and compositions of the disclosure to promote periodontal and osseous repair or regeneration in a hyperglycemic or diabetic subject can be determined using various clinically available methods such as with various spectroscopic and/or imaging techniques, histologic and immunohistochemical assays and techniques, and the like available in the art.

The biocompatible material (e.g., alloplastic, allogeneic and/or xenogeneic bone) comprising PDGF, will comprise an effective amount of PDGF to promote bone repair. As used herein, the term “effective amount” means the amount of the PDGF or PDGF and another biologically active agent that will elicit an efficacious biological or clinical response in a subject in need thereof.

As used herein, the term “subject” comprises a mammal. In some embodiments, the subject has hyperglycemia and/or diabetes. Exemplary mammals include: primates, such as monkeys, apes, and humans; pigs, cows, and other livestock; domesticated pets, such as dogs and cats; and other animals, such as horses. Typically, the subject is a human. Hyperglycemia and/or diabetes inhibits proper bone regeneration and/or repair in such subjects. More particularly, hyperglycemia and/or diabetes inhibit maxillofacial bone regeneration and/or repair. Similarly, hyperglycemia and/or diabetes inhibit or negatively impact periodontal bone regeneration and/or repair. Accordingly, the methods and compositions of the disclosure are particularly suited to treating such subjects having hyperglycemia and/or diabetes.

In certain embodiments, the disclosed methods can further comprise delivering or adding additional PDGF (e.g., PDGF-BB) directly to the site of injury/repair alone or in combination with a biocompatible bone matrix material after implantation. The additional PDGF can be delivered or added 1, 2, 3, 4, 5, 6, 7 days after implantation of the biocompatible material comprising PDGF (e.g., PDGF-BB). In certain embodiments, the additional PDGF can be delivered or added once during surgery or can be added once per week or several times per week after its application to the injured site to improve gingival and periodontal tissues repair and/or regeneration. In some embodiments, a clinician can delivery or add additional PDGF to the biocompatible bone matrix material at suitable intervals and for a duration depending upon bone regeneration or repair at the site of implantation.

The disclosure demonstrates the efficacy of using alloplastic, allogeneic and/or xenograft biomaterial soaked in recombinant human PDGF (rhPDGF) for GBR in periodontal defects in diabetic subjects or subject with hyperglycemia. Histological and μCT analysis showed the use of rhPDGF in adjunct with bone graft biomaterial and an optional collagen membrane resulted in regenerating the buccal, radicular, interradicular, furcal bone and periodontal intrabony/infrabony/vertical/horizontal defect. Although the mechanism of action is not required, an explanation in this regard may be associated with the biological properties of rhPDGF. rhPDGF exhibits stimulatory effects (as a chemo-attractant and mitogen) on mesenchymal cells (including osteogenic cells) and is also known to promote angiogenesis. It has also been reported that PDGF stimulates osteoblast type-I collagen and osteopontin expression in vitro and osteopontin expression in vivo.

The findings presented herein and below support the hypothesis that local application of biocompatible impregnated PDGF or particulate graft material comprising rhPDGF promotes osteogenic cell proliferation in osseous defects thereby leading to a subsequent increase in the bone regeneration. Another factor that can play a role in forming new bone periodontal osseous defects in the present study is the ability of rhPDGF to promote angiogenesis through up-regulation of vascular endothelial growth factor (VEGF). The VEGF is expressed by proliferating osteoblasts and hypertrophic chondrocytes and is a major regulator of endochondral bone formation.

A biocompatible material used in the disclosure may be of any shape to provide proper bone formation. In some embodiments, pores or spaces in the material can be adjusted by one of skill in the art to allow adsorption or absorption, and/or allow or prevent migration of cells into or through the matrix material once implanted.

The invention has been generally described above and is further exemplified by the following examples, which are intended to illustrate but not limit the invention.

### Examples

Six diabetic male beagle dogs with a mean age and weight of 12±0.4 months and 13±1.2 kilograms (Kg) respectively, were used. Subjects were distributed evenly and randomly to one of two groups: (1) rhPDGF group (G1) (N=3), and (2) Emdogain group (G2) (N=3). Diabetic canines were monitored closely for their glycemic level (three times/day). All surgical and non-surgical procedures were performed under general anesthesia using ketamine (Pfizer Limited, Sandwich, Kent, CT13 9NJ, UK) (10 milligrams [mg]/Kg body-weight) with buccal infiltration of xylocaine (AstraZeneca LP for DENTSPLY Pharmaceutical, York Pa.). Non diabetic dogs control group were based upon previous publications.

Induction of experimental diabetes and preoperative management. Three dogs were fasted for 24 hours before the induction of diabetes via intravenous injection of STZ. Blood was collected for baseline levels of fasting plasma glucose. Since there was a risk of fatal hypoglycemia as a result of massive pancreatic insulin release, dogs were treated with 10% glucose solution after 4 hours of diabetes induction. After five days, the dogs had fasting hyperglycemia (glucose millimoles/Liter). Under general anesthesia, all animals received supragingival scaling one week prior to extraction and once/month following implant placement using an ultrasonic scaler (NSK, Westborough, Mass.). IM antibiotics (Ampicillin 25 mg/Kg body weight) were administered a day before and at the time of surgery. Antibiotics were continued for 3 days after surgery as 25-50 mg/Kg IM every 8 hours.

Dehiscence and chronic periodontal defect induction and treatment. Preoperative intra-muscular (IM) amoxycillin (25 mg/Kg body weight) (Betamox LA. Norbrook Laboratory Limited, Newry, County Down, Northern Ireland) were administered at the day of surgery. Following general anesthetic (GA) (as described previously), subjects were draped, and the surgical sites were swabbed with an antiseptic solution (The Purdue Fredrick Company, Stamford, Conn., USA). Local anesthesia (Astra, Westborough, Mass., USA) was administered and full thickness mucoperiosteal flaps were raised. Standardized dehiscence and a three-walled periodontal bony defect (3 mm×5 mm) involving the interdental bone was created on the mesial walls of the second premolars (P2) using high speed round bur and hand chisel. A primary closure of the defect was achieved using vicryl suture 5-0 (Ethicon, Johnson &amp; Johnson Medical N.V., Belgium). Total of 4 defect per Canine and total of 12 defects for each treatment group.

Twelve-weeks after defect induction, preoperative antibiotics (as described above) were readministered to all subjects for the second regenerative surgery. Full thickness mucoperiosteal flaps were raised under GA to expose the induced chronic periodontal bony defects. Defect sites were randomly assigned as “G1” and “G2”. Randomization was performed by picking a paper from a brown bag. Each group has 8 defect sites were treated with particulate graft material (Puros® cortical particulate allograft, Zimmer dental, Carlsbad, Calif., USA) soaked in: (1) G1:0.5 ml of 0.3 mg/ml rhPDGF (GEM 21S®, Osteohealth, Shirley, N.Y., USA); (2) Emdogain (EMD) (Straumann AG, Basel, Switzerland). Primary closure was achieved using vicryl suture 5-0 (Ethicon, Johnson &amp; Johnson Medical N.V., Belgium).

Post-operative management. All animals received IM injections of amoxicillin (25 mg/Kg body weight) (Betamox LA. Norbrook Laboratory Limited, County Down, Northern Ireland) at every 8 hours for 5 days. IM Analgesics (0.01-0.02 mg/Kg) (Buprenorphine, Idaho Falls, Id., USA) were administered immediately after surgery and every 8 hours for the first two days after surgery and then whenever needed depending upon the presence of signs of pain by the animal (such as restlessness, unusual calmness, and refusal to eat). Plaque control procedures, using non-scaling and root planing and topical application of a 0.2% chlorhexidine digluconate solution (GUM, Chicago, Ill., USA) were performed once weekly for 4 months after surgery. Vicryl suture 5-0 (Ethicon, Johnson &amp; Johnson Medical N.V., Belgium) were removed after 10 days of surgery.

Measurement of periodontal parameters and non-surgical periodontal therapy. Periodontal parameters (Plaque index [PI], bleeding on probing [BOP] and probing depth [PD] were measured after 4 weeks of induction of periodontal bony defect. PD was measured using a graded probe. Periodontal parameters were measured on six sites (mesiobuccal, midbuccal, distobuccal, mesiopalatal/lingual, midpalatal/lingual, and distopalatal/lingual) around teeth. Non-surgical periodontal therapy (using an ultrasonic scaler) was performed weekly after treatment of the defects until euthanasia. Fasting Blood Glucose (FBG) levels of animals were also measured.

Euthanasia, jaw sectioning and μCT examinations. After 16-weeks, all subjects were sacrificed under GA (as described previously) with an intravenous overdose of 3% sodium pentobarbital (WA Butler Company, Dublin, Ohio, USA). FBG levels of animals were also measured.

The jaw segments containing teeth with induced dehiscence defects and associated mesial and distal tooth structures were removed en block using an electric saw (Leica SP 1600, Bannockburn, Ill., USA) and fixed in 10% neutral formalin solution. μCT scanner (SkyScan 1172, CT-Analyser, Version 1.11.4.2+, Kontich, Belgium) was used to evaluate the BBT, BBV and vertical bone height (VBH) around teeth with dehiscence defects. The x-ray generator of the μCT was operated at an accelerated potential of 101 kilo-volts with a beam current of 96 micro-amperes using an aluminum filter with a resolution of 37.41 micrometer pixels. The buccal bone thickness (BBT) was measured at every 1-mm level starting from alveolar crest till the base of the defect. The buccal bone volume (BBV) was measured as described in previous study. In the adjacent teeth, vertical bone height (VBH) was measured using linear measurements (in mm), which extended from the crest of the cementoenamel junction of the tooth to the apical notch.

Light Microscopy. Jaw segments were decalcified in a solution containing equal parts of 50% formic acid and 20% sodium citrate for 10 weeks. The decalcified specimens were washed in running water, dehydrated in an ascending ethanol series and embedded in paraffin. Polymerized blocks were primarily ground in order to bring the tissue components closer to the cutting surface. A section of 100 micrometer (μm) thickness attached to the second slide was cut by a diamond blade saw and under a pressure of 50 grams (g) to 100 g. An ultimate thickness of 40 μm was attained by grinding and polishing with 1200-, 2400- and 4000-grit sandpaper, and each block sections were stained with Toluidine blue/pyronin G 18.

Statistical analysis. Statistical analysis (SPSS version 18. IL) was performed using a software program. BBT, BBV and VBH between the groups were assessed using one way analysis of variance. P-values less than 0.05 were considered statistically significant.

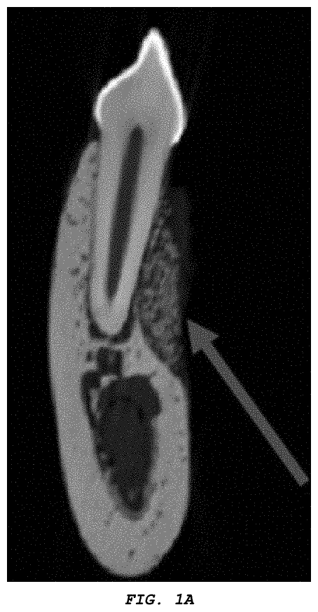

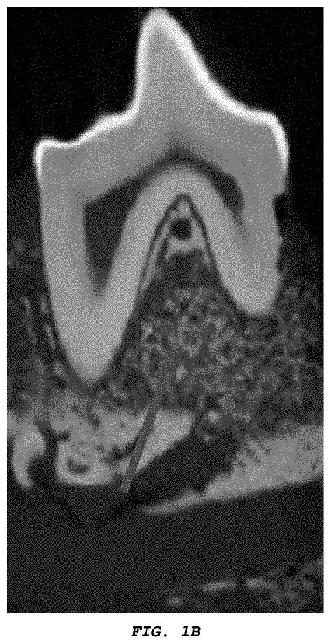

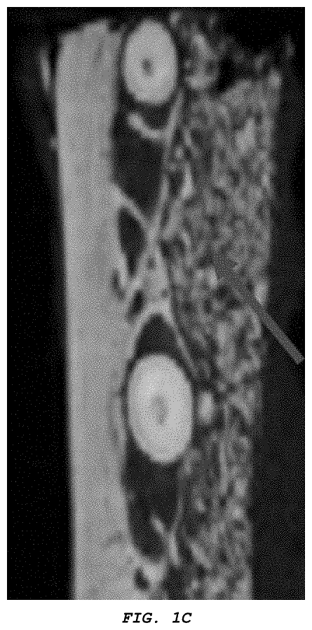

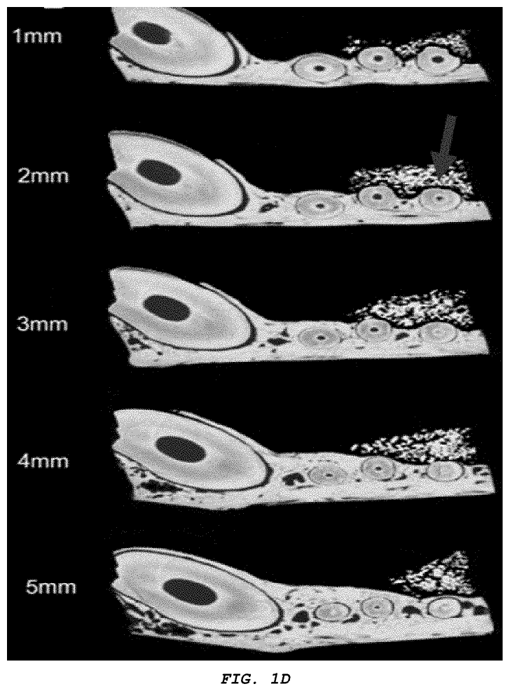

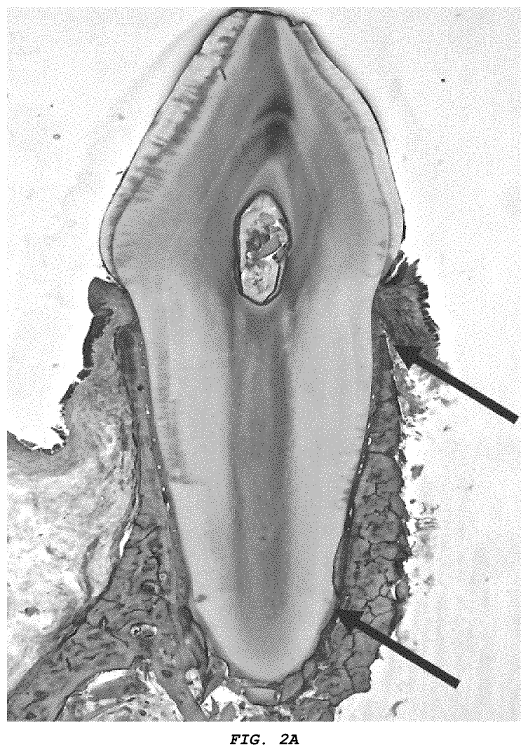

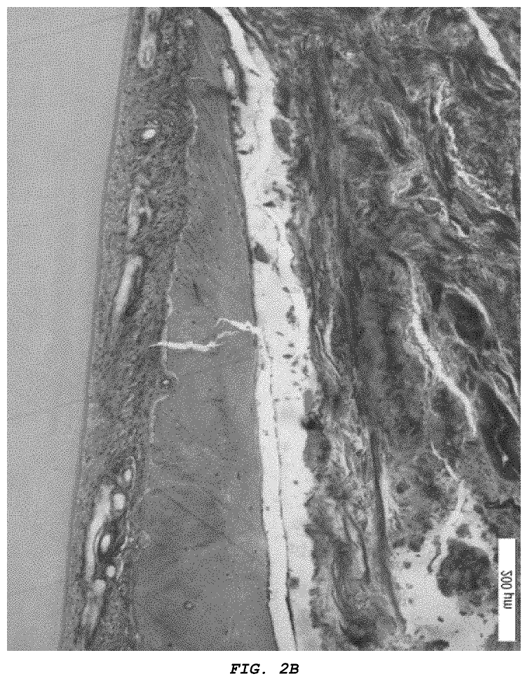

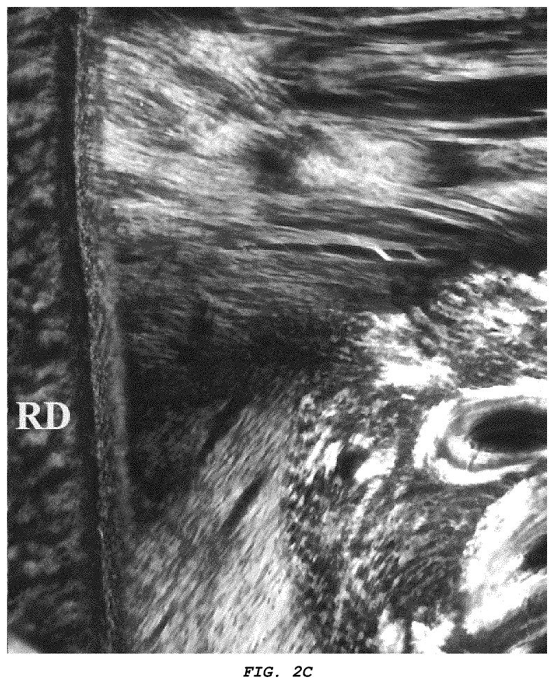

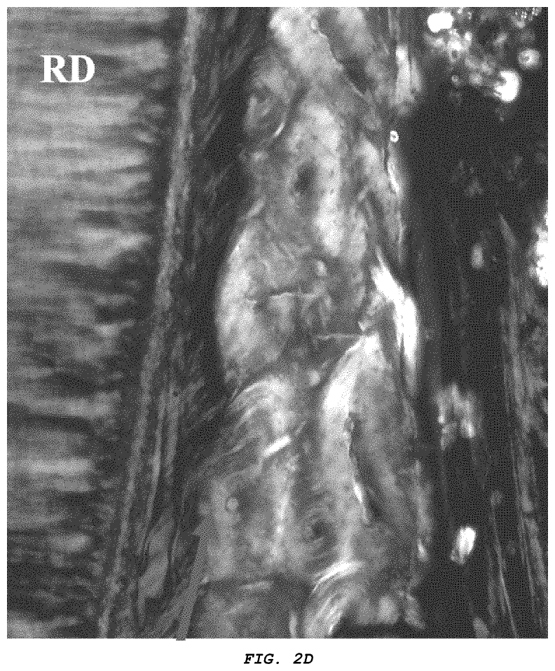

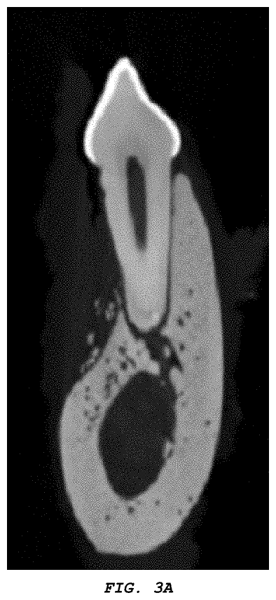

rhPDGF treated sites. μCT measurements 1.09±0.9 mm, 22.3±9.3 mm³ and 3.5±1.09 mm for BBT, BBV and VBH, respectively (P<0.05) (FIG. 1, Table 1). 3D μCT reconstructed images shows: (1) buccal bone significant thickness and its vertical height (FIG. 1A), (2) mid-sagittal section 3D demonstrating the fill of the 3 wall defect with bone, (3) axial section at middle apico-coronally demonstrating consistent bone thickness and interproximal bone fill (FIG. 1C) and (4) axial section at 1 mm corono-apically showing evidence of bone thickness through the entire axial sections. Light microscopy examination shows the hitmorphometirc measurements were similar to μCT measurements in the BBT and VBH. BBT was measured from the base of the notch (FIG. 2A) to the most coronal part of buccal bone (FIG. 2A-B). The majority of the regenerated sites demonstrated no clear transition between basal bone and augmented area could be detected, but the new bone demonstrated higher density in the crestal area. In all specimens a functional attachment was evident via the vertical connective tissues/fibers insertion into the cementum and the connective tissue vertical organization between regenerated bone and cementum. (FIG. 2C-D). All periodontal defect regenerated interproximally with bone.

TABLE 1
Buccal bone thickness, buccal bone volume and vertical bone
height following guided bone regeneration with and without
adjunct platelet derived growth factor therapy in dogs
with and without streptozotocin-induced diabetes.

| | rhPDGF | EMD |
|---|---|---|
| Parameters | (n = 12) | (n = 12) |
| Buccal bone thickness (in mm) | 1.09 ± 0.9* | ±0.08^{§}0.3 |
| Buccal bone volume (in mm³) | ±9.3^{†}22.3 | 8.8 ± 1.5^{¶} |
| Vertical bone height (in mm) | ±1.09^{‡}3.5 | ±0.06^{#}0.2 |

*P < 0.05
^{†}P < 0.05
^{‡}P < 0.05
^{§}P < 0.05
^{¶}P < 0.05
^{∥}P < 0.05

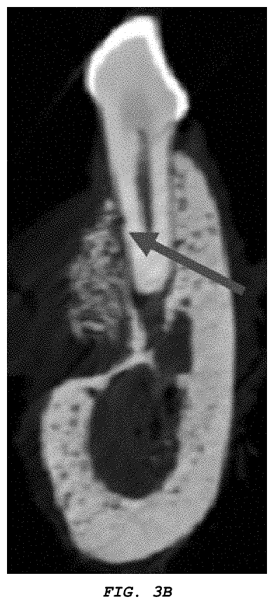

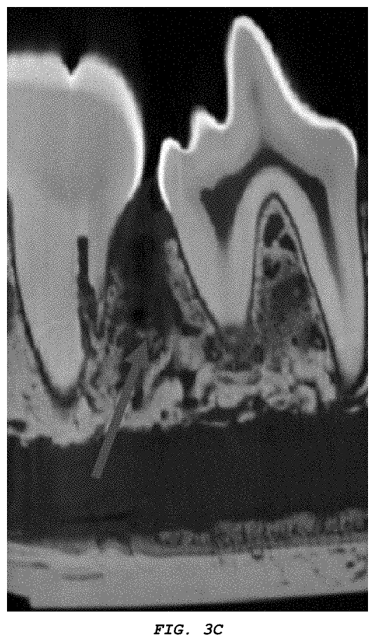

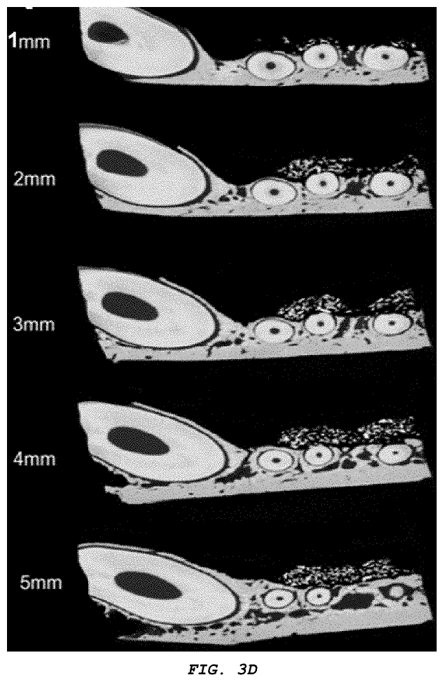

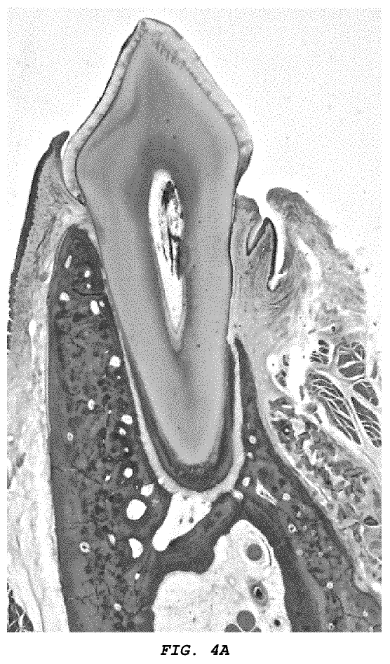

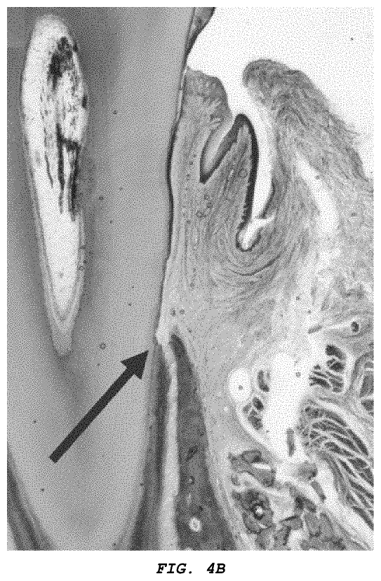

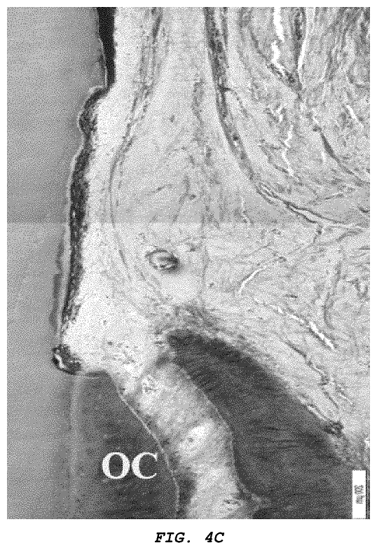

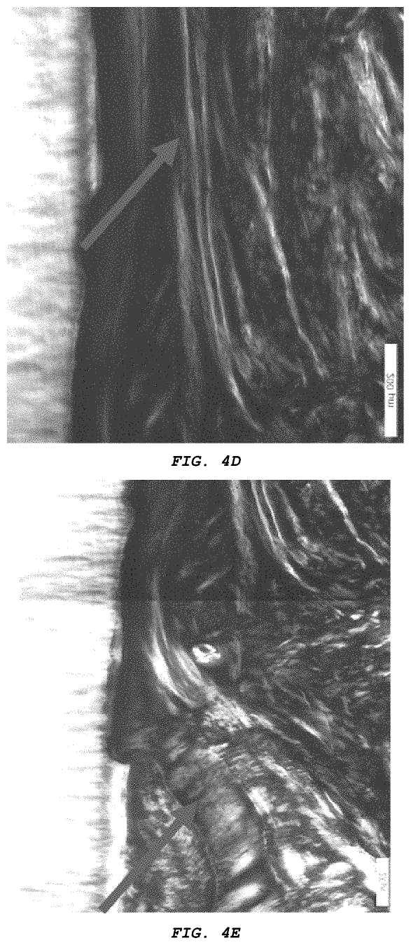

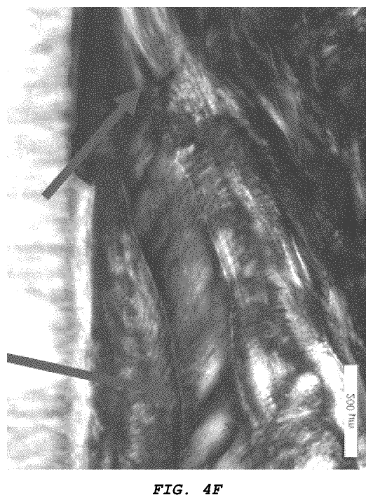

EMD treated sites. μCT measurements BBT, BBV and VBH were 0.3±0.1 mm, 8.8±1.5 mm³ and 0.2±0.06 mm, respectively. Periodontal intrabony defect did not demonstrate bone regeneration. 3D μCT reconstructed images shows: (1) no buccal bone was regenerated (FIG. 3A), (2) only 1 mm of bone regenerated buccal bone coronal to the notch (FIG. 2B), (3) interproximal bone resorption with no sign of bone regeneration, and (4) axial section at 1 mm each, demonstrate the first 4 mm had complete absence of bone or very thin bone thickness (FIG. 3D). Dehiscence buccal bone did not yield in regeneration in majority of the site and only two cases where bone regeneration was evident but did not grow vertically beyond 1-2 mm coronal to the basal notch. A long junctional epithelium type of attachment and a functional parallel connective tissue fiber between gingival mucosa and root dentin. Interestingly, one case demonstrated afunctional parallel fibers (FIG. 4D) where the remaining samples showed vertical functional fibers insertion (FIG. 4E-F).

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising alloplastic, allogeneic and/or xenogeneic bone graft material comprising recombinant human platelet derived growth factor-BB (rhPDGF-BB).

2. The composition of claim 1, wherein the bone graft material is xenogeneic to a subject.

3. The composition of claim 1, wherein the bone graft material is allogeneic to a subject.

4. The composition of claim 1, wherein the bone graft material is alloplastic to a subject.

5. The composition of claim 1, wherein the alloplastic, allogeneic and/or xensogeneic bone graft material comprising rPDGF further comprises one or more additional factors.

6. The composition of claim 5, wherein the one or more additional factors comprise extracellular matrix materials.

7. The composition of claim 6, wherein the extracellular matrix materials are selected from the group consisting of Type I collagen, Type II collagen, Type III collagen, bovine collagen, human collagen, porcine collagen, equine collagen, avian collagen, fibronectin, alginate and any combination thereof.

8. The composition of claim 5, wherein the one or more additional factors are growth factors.

9. The composition of claim 5, wherein the one or more additional factors are antibiotics, antifungals or a combination thereof.

10. A method of treating a bone and/or tissue injury in a subject with hyperglycemia, comprising administering a composition of claim 1.

11. The method of claim 10, wherein the bone and/or tissue injury is at a site surrounding or associated with a tooth or teeth.

12. The method of claim 10, wherein the subject has diabetes.

13. The method of claim 12, wherein the diabetes is type 1 or 2 diabetes.

14. The method of claim 10, wherein the bone injury is the result of a periodontal infection.

15. The method of claim 10, wherein the bone injury is a maxillofacial bone injury, pathology, and/or tumor.

16. The method of claim 15, wherein the bone injury is a periodontal bone injury.

17. The method of claim 15, wherein the periodontal bone injury surrounds or abuts a tooth.
